(19) United States
(12) Patent Application Publication
(10) Pub. No.: US 2024/0382319 A1
(43) Pub. Date: Nov. 21, 2024

(54) EXPANDABLE INTERVERTEBRAL CAGE

(71) Applicant: 25SEGMENTS AG, Zürich (CH)

(72) Inventors: Daniel FLURI, Grenchen (CN); Mazda FARSHAD, Zumikon (CH); Robert FRIGG, Bettlach (CN); Patrick BURKI, Solothum (CH); Marcel AESCHLIMANN, Zuchwil (CH)

(73) Assignee: 25SEGMENTS AG, CH-8008 ZURICH (CH)

(21) Appl. No.: 18/686,304

(22) PCT Filed: Aug. 10, 2022
(86) PCT No.: PCT/EP2022/072411

§ 371 Date: Feb. 23, 2024

(30) Foreign Application Priority Data
Aug. 25, 2021 (CH) CH070201/2021

(51) Int. Cl. A61F 2/44; A61F 2/30; A61F 2/46

(52) U.S. Cl. CPC A61F 2/447; A61F 2/4611; A61F 2002/30476

(57) ABSTRACT

The present disclosure relates to an expandable intervertebral cage (1) with a base (2) comprising a peripheral circumferential side wall (7) extending in a first direction (x) and a first bone interaction surface (5); a stage (3) comprising a second bone interaction surface (6) arranged essentially opposite to the first bone interaction surface (5) with respect to the expandable intervertebral cage (1); and a locking mechanism (4) for locking the position of the second bone interaction surface (6) of the stage (3) with respect to the first bone interaction surface (5) of the base (2) at least in the first direction (x) and being arranged at least partially within the circumferential side wall (7) of the base (2).

### BACKGROUND OF THE INVENTION

### Field of the Invention

The present disclosure relates to an expandable intervertebral cage. The expandable intervertebral cage, hereinafter also referred to as cage, can be brought as an implant into an intervertebral space in a retracted state, and once in place, can be expanded to a desired expanded state.

### Discussion of Related Art

The present disclosure relates to an expandable intervertebral cage. The expandable intervertebral cage, hereinafter also referred to as cage, can be brought as an implant into an intervertebral space in a retracted state, and once in place, can be expanded to a desired expanded state.

### SUMMARY OF THE INVENTION

The surgical challenge of placement of an intervertebral cage is usually the limited available approach space due to bony structures, such as the endplates or the facet joints, and nervous structures, such as the dural sac and nerve roots, for dorsal approaches, and additionally by vascular structures for anterior or lateral approaches. The most commonly used approaches to place a cage are dorsal in nature, and often referred to as posterior lumbar interbody fusion (PLIF) or lateral lumbar interbody fusion (LLIF). For PLIF approaches, the nerve root and the dural sac are retracted medially to be able to open the disc space to allow removal of the disc before inserting the intervertebral cage. An intervertebral cage has thus very limited dimensions in order to fit into said disc space. Furthermore, once fitted the intervertebral cage needs to be accessed (usually one sided) in order to expand the cage. The challenge to insert a lateral lumbar interbody fusion cage (LLIF) lies in the limited access space in the soft tissue structures and the difficulty to create a sufficient interbody space to place an optimal sized cage without damaging the endplates of the vertebral bodies.

As mentioned above, expandable intervertebral cages are known from the prior art, however, these devices typically have a rather complex structure and there is often a risk of accidentally collapsing them, when in the intervertebral space, as they are not self-stable.

The present disclosure overcomes these problems by providing a simple but robust and versatile intervertebral cage with a shallow design that allows the cage to be inserted into the intervertebral disc space in a minimally invasive manner through a comparatively small opening with respect to the prior art.

The present disclosure relates to an expandable intervertebral cage with a base and a thereto displaceable stage. The base comprises a peripheral circumferential side wall extending in a first direction and having a first bone interaction surface. The stage comprises a second bone interaction surface arranged essentially opposite to the first bone interaction surface with respect to the expandable intervertebral cage. For a good fusion capability, the first bone interaction surface and/or the second bone interaction surface may circumvent at least partly an opening to a filling room for accommodating bone graft. The base and/or the stage can comprise multiple parts connected to each other.

Depending on the application, the stage can be only displaced with respect to the base in a first direction. In that case, the first bone interaction surface and the second bone interaction surface can be arranged at a fixed angle with respect to each other such that the lumbar angle of the vertebrae can be taken into account. If an individual adjustment of the lumbar angle is needed, the stage may however be additionally tilted around a further direction. Depending on the application of the cage (e.g. LLIF or PLIF) the stage can either tilt around a second or a third direction. In the context of this disclosure the first, second and third direction are essential perpendicular to each other. For lateral lumbar interbody fusion (LLIF) applications, the stage is advantageously tiltable with respect to the base around the second direction. Meanwhile, for posterior lumbar interbody fusion (PLIF) applications, the stage is preferably tiltable with respect to the base around the third direction. The second and third direction may thereby be further defined as follows: If the cage has essentially the form of a rectangular prism, the circumferential side wall can be divided into a first, a second, a third and a fourth side wall, wherein the first and the second side walls are shorter than the third and the fourth side walls. The third and the fourth side walls can then extend in said second direction. Consequently, the first and the second side walls can extend in said third direction. Thus, for lateral lumbar interbody fusion applications the stage preferably tilts around the second direction parallel to the third and fourth wall, meanwhile for posterior lumbar interbody fusion applications the stage can tilt around the third direction parallel to the first and second wall.

For secure positioning and expansion, the cage can be expanded with the help of a tool, instead of housing a complex expansion mechanism within the cage. Such a tool can be inserted into the cage and serves to expand the cage from the retracted state to at least one expanded state by lifting and/or tiling the stage with respect to the base. However, for a fine adjustment, the cage can be expanded from the retracted state to multiple (intermediate) expanded states, until a fully expanded state is reached. Advantageously, the cage can be expanded from a minimal height of 5-13 mm in the retracted state to a maximum height of 9-19 mm in the fully expanded state. Furthermore, if tilting is intended, the tilting angle may be in the range from 0 to 15° degree. However, even though the expansion can be performed by an external tool, the cage has to provide a looking mechanism to lock the cage in the at least one expanded state (such as the intermediate expanded state(s) and/or the fully expanded state) and thus avoid accidentally collapsing of the cage. Therefore, the locking mechanism preferably comprises at least one latch arranged in a displaceable manner, in particular in the second direction, between a locked position and an unlocked position with respect to the first direction. Hereby, the stage is supported and locked in the first direction with respect to the base in the locked position. Meanwhile, in the unlocked position, the at least one latch is displaceable in the first direction with respect to the base. Furthermore, in the unlocked position the base and the stage can be disengaged and thus disassembled. Depending on the application the number of latches may differ. Advantageously, the latch is arranged within the circumferential side wall of the base in the locked position and the unlocked position. If the stage has to be only lifted with respect to the base in the first direction, one latch is sufficient. If the stage should further be tiltable, also two latches (e.g. a first and a second latch) can be used.

If only one latch is used, it may however be advantageous if the latch is frame-like comprising a first, a second, a third and a fourth inner wall, wherein the first and the second inner walls are shorter than the third and the fourth inner walls. In a mounted position the first inner wall can be arranged adjacent to the first side wall of the base, the second inner wall is arranged adjacent to the second side wall of the base, and so on. For a good fusion capability, the frame-like latch may circumvent a filling room for accommodating bone graft.

Independently of the application, the at least one latch preferably comprises at least one first tooth configured to latch in the locked position with at least one second tooth arranged at the base. However, for a fine adjustment in height (in the first direction) and/or tilting angle, multiple first and/or second teeth are advantageous. The multiple first and/or second teeth are preferably arranged in a row, wherein the multiple teeth of the respective row are spaced apart from each other in the first direction. Hereby, at least one row of multiple first teeth can be arranged on an outer side of the at least one latch. The outer side of the latch hereby faces an inner side of the circumferential side wall of the base in a mounted position. For a frame-like latch, the rows of first teeth can e.g. be arranged opposite of each other with respect to the latch on the third and the fourth inner walls. Accordingly, at least one row of multiple second teeth may be arranged on the inner side of the circumferential side wall (in particular the third and the fourth side walls). Preferably, a groove extending in the first direction is arranged adjacent to the row of the second teeth on said inner side. Hence, in the second direction the row of second teeth is arranged adjacent to the respective groove. Also, on the outer side of the latch, a respective groove may be arranged adjacent to the row of first teeth. Thus, a groove and a row may be arranged alternating next to each other on the respective inner and/or outer side. By displacing the at least one latch in the second direction, the first teeth (respectively the at least one row of first teeth) of said latch disengage from the second teeth (respectively the at least one row of second teeth). In the unlocked position the first teeth (respectively the at least one row of first teeth) are then arranged in the respective groove of the circumferential side wall. Additionally, or alternatively, the second teeth (respectively the at least one row of second teeth) are arranged in the respective groove of the latch. Thus, no undercut is formed between the latch and the base in the first direction in said unlocked position. In case that the cage has essentially the form of a rectangular prism, the second teeth, respectively the row of second teeth, may be arranged only on the third and fourth side walls. Thereby pairwise opposite arranged rows of second teeth are advantageous for the overall stability of the cage. Thus, preferably, at least one row of second teeth is arranged on the third side wall opposite of a corresponding row of the second teeth arranged on the fourth side wall. If only one latch is used, also the rows of the first teeth of the latch can be arranged pairwise opposite of each other with respect to the latch.

In order to move the at least one latch from the locked to the unlocked position, the at least one latch may comprise a thread extending in the second direction into the latch. A screw extending from the base or from the stage into latch can further engage with the thread. By operating the screw, the respective latch is displaced in the second direction with respect to the base, thereby displacing the first teeth from the locked position to the unlocked position. Preferably, the screw is operateable through on opening arranged on the first side wall of the circumferential side wall. Furthermore, a tool opening for receiving the tool to expand the cage can be arranged on said first side wall. Preferably, the tool opening is arranged centered on the first side wall in the third direction. Once the tool is inserted, the tool may engage in the cage with a tool interface (also preferably arranged centered of the cage in the third direction) for temporarily affixing the tool during operation, respectively during the expansion of the cage.

As explained above, the expandable intervertebral cage can comprise more than one latch, in particular a first latch and second latch. Having two latches allows to tilt the stage with respect to the base. Therefore, the first latch and second latch can be arranged independently displaceable with respect to each other in the second direction from a respective locked position to an unlocked position. In the unlocked position the respective latch in said position is additionally displaceable in the first direction. The hereinafter in more detail described locking mechanism comprising two latches displaceable in the second direction from the locked to the unlocked position is thus particularly suitable for lateral lumbar interbody fusion applications, whereby the stage is tiltable around the second direction parallel to the third and fourth wall. The first latch can hereby extend along the third side wall and the second latch can extend along the fourth side wall of the circumferential side wall of the cage. In that case, the first teeth of the first latch can engage in the second teeth arranged on said third side wall in the locked position of the first latch and the first teeth of the second latch can engage in the second teeth arranged on the fourth side wall in locked position of the second latch. As explained above, each latch may have a thread for displacing the respective latch independently from the other. Thus, two screws for operating the respective latches can be arranged opposite of each other with respect to the tool opening (extending through the first side wall). As explained above, the tool opening serves for inserting the tool into the cage such that the tool can be interconnected to the tool interface of the cage.

If two latches are present, the tool interface is preferably arranged between the first and the second latch in the third direction. Thus, in the unlocked position the tool can support the first and the second latch to avoid that the latches disconnect from the base. Meanwhile, in the locked position, the first latch and the second latch can support each other in a third direction and thus avoid that the latches disconnect from the base if no tool is present. Therefore, the first latch can comprise a first support and the second latch can comprise a second support. In the locked position the first and second support can then be braced against each other in the third direction. For a space saving design it is advantageous, if the first and the second support are arranged in the mounted position opposite of the tool opening. Thus, the first and the second support can be arranged adjacent to the second side wall of the circumferential side wall.

Instead of two latches also other mechanisms can be used for tilting. It is e.g. possible that the latch and the stage can be displaced together (in the unlocked position) with respect to the base in the first direction (allowing an adjustment in height of the cage) and the stage can be tilted independently from the latch and the base (allowing an adjustment in the patient's lumbar angle). Therefore, the intervertebral cage may e.g. comprise a rotatable camshaft configured to tilt the stage with respect to the base and the latch. Preferably the camshaft extends in the second direction. Thus, by rotating the camshaft a cam lifts the stage from the latch on one side of the cage, such that the stage is tilted with respect to the base (and the latch). The camshaft may be mounted in the latch and/or a mounting frame, as described in more detail below. Therefore, the latch may have at least one opening or recess for mounting of the camshaft. For this application, it may be advantageous if the latch is further frame-like, as described above. The camshaft may then be arranged adjacent to the third inner wall of the latch, wherein the below explained linear guiding structure can be arranged on the fourth inner wall of the latch interconnecting the latch and the stage.

Alternatively, the intervertebral cage may also comprise a displaceable wedge to facilitate tilting of the stage with respect to the latch and the base. The wedge can be displaceable in either the second or the third direction. The wedge may be arranged between the latch and the stage in the first direction. The wedge comprises a first gliding surface and a second gliding surface. The first and the second gliding surfaces are thereby angled with respect to each other in a displacement direction of the wedge. During displacement, the first gliding surface interacts with a contact surface of the stage and the second guiding surface interacts with a contact surface of the latch. Thus, by displacing the wedge, the stage is lifted locally (one sided of the cage) with respect to the base in the first direction resulting in a tilting movement of the stage with respect to the base (and the latch). Also, for this application it may be advantageous if the latch is frame-like, as described above. The wedge may then be arranged between the third inner wall and the stage, wherein the below explained linear guiding structure can be arranged on the fourth inner wall.

For a reliable displacement, the expandable intervertebral cage can further comprise at least one linear guiding structure interconnecting the at least one latch to the stage and configured to support the displacement of the at least one latch in the second direction. If two latches are present, two independent linear guiding structures per latch are advantageous. Depending on the application, the respective linear guiding structure may be configured to allow a rotation of the latch with respect to the stage around the second direction. This is especially useful, if tilting is intended. E.g., the at least one linear guiding structure may comprise a rod extending in the second direction inter-connecting the stage with the respective latch.

To further reduce unwanted displacement of the base with respect to the stage in the second direction, the cage may comprise at least one strut extending in a mounted position from the stage into the circumferential side wall of the base.

If no tilting is intended, the at least one latch can be mounted displaceable in the stage. However, for tiling applications, the cage can comprise a mounting frame for mounting the latch displaceable in the second direction. The mounting frame may be arranged at least partially within the circumferential side wall of the base. The mounting frame can thereby be braced against the circumferential side wall of base. For mounting the latch in the mounting frame at least one rod can extend at least partially through the latch and in the mounting frame. Furthermore, the latch may be interconnected by the linear guiding structure to the stage allowing the stage to tilt with respect to the latch and the base. Thus, the latch and the stage can be displaced together in the first direction (allowing an adjustment in height of the cage) and the stage can be tilted independently from the latch and the base (allowing an adjustment in the patient's lumbar angle).

It is to be understood that both the foregoing general description and the following detailed description present embodiments and are intended to provide an overview or framework for understanding the nature and character of the disclosure. The accompanying drawings are included to provide a further understanding, and are incorporated into and constitute a part of this specification. The drawings illustrate various embodiments, and together with the description serve to explain the principles and operation of the concepts disclosed.

### BRIEF DESCRIPTION OF SEVERAL VIEWS OF THE DRAWINGS

The herein described invention will be more fully understood from the detailed description given herein below and the accompanying drawings which should not be considered limiting to the invention described in the appended claims. The drawings are showing:

FIG. 1 shows a first variation of the expandable intervertebral cage in a perspective view;

FIG. 2 shows the first variation of the expandable intervertebral cage according to FIG. 1 in a top view;

FIG. 3 shows a sectionized view A-A of the expandable intervertebral cage according to FIG. 2;

FIG. 4 shows the first variation of the expandable intervertebral cage according to FIG. 1 in a disassembled exploded view;

FIG. 5 shows a second variation of the expandable intervertebral cage in a perspective view;

FIG. 6 shows the second variation of the expandable intervertebral cage according to FIG. 5 in a top view;

FIG. 7 shows a sectionized view B-B of the expandable intervertebral cage according to FIG. 6 in a retracted state;

FIG. 8 shows a sectionized view B-B of the expandable intervertebral cage according to FIG. 6 in an expanded state;

FIG. 9 shows the second variation of the expandable intervertebral cage according to FIG. 5 in a disassembled exploded view;

FIG. 10 shows a third variation of the expandable intervertebral cage in a disassembled exploded view; and

FIG. 11 shows a fourth variation of the expandable intervertebral cage in a disassembled exploded view.

### DETAILED DESCRIPTION OF THE INVENTION

Reference will now be made in detail to certain embodiments, examples of which are illustrated in the accompanying drawings, in which some, but not all features are shown. Indeed, embodiments disclosed herein may be embodied in many different forms and should not be construed as limited to the embodiments set forth herein; rather, these embodiments are provided so that this disclosure will satisfy applicable legal requirements. Whenever possible, like reference numbers will be used to refer to like components or parts.

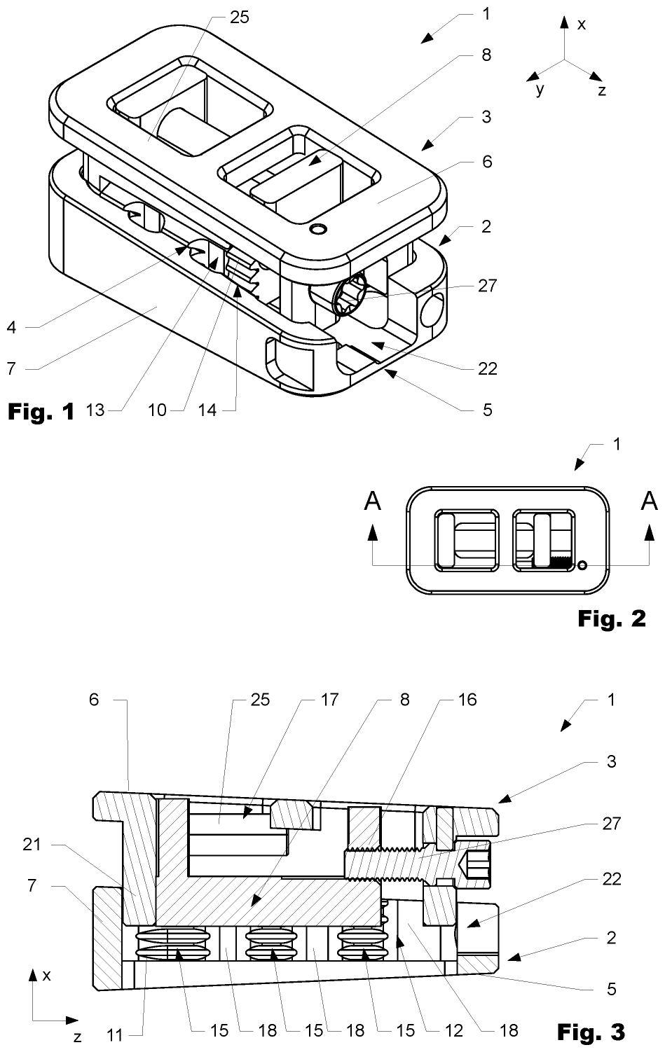

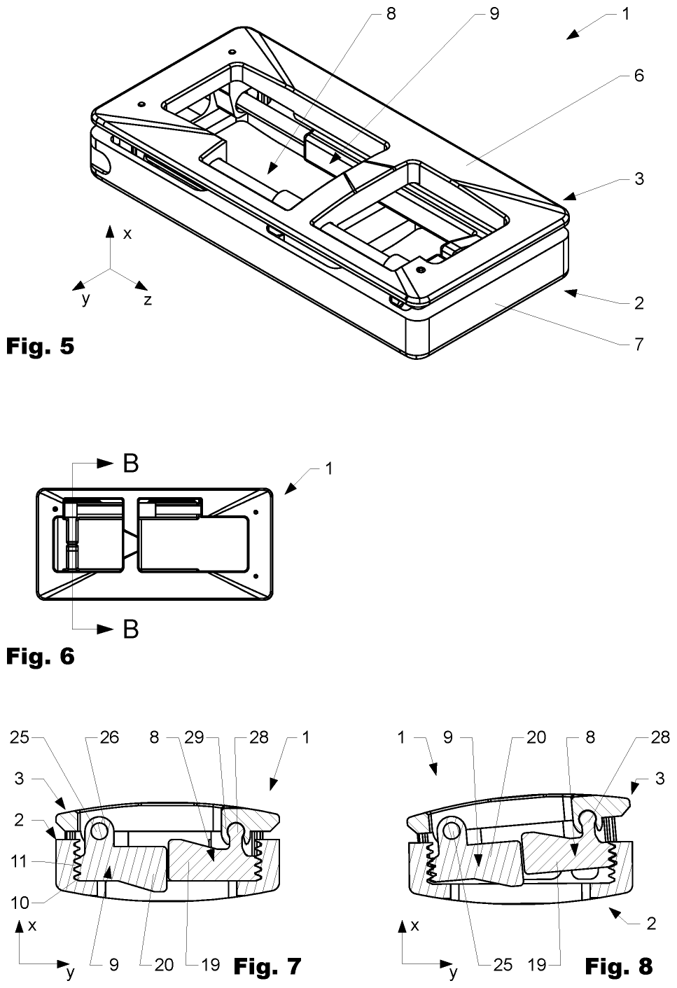

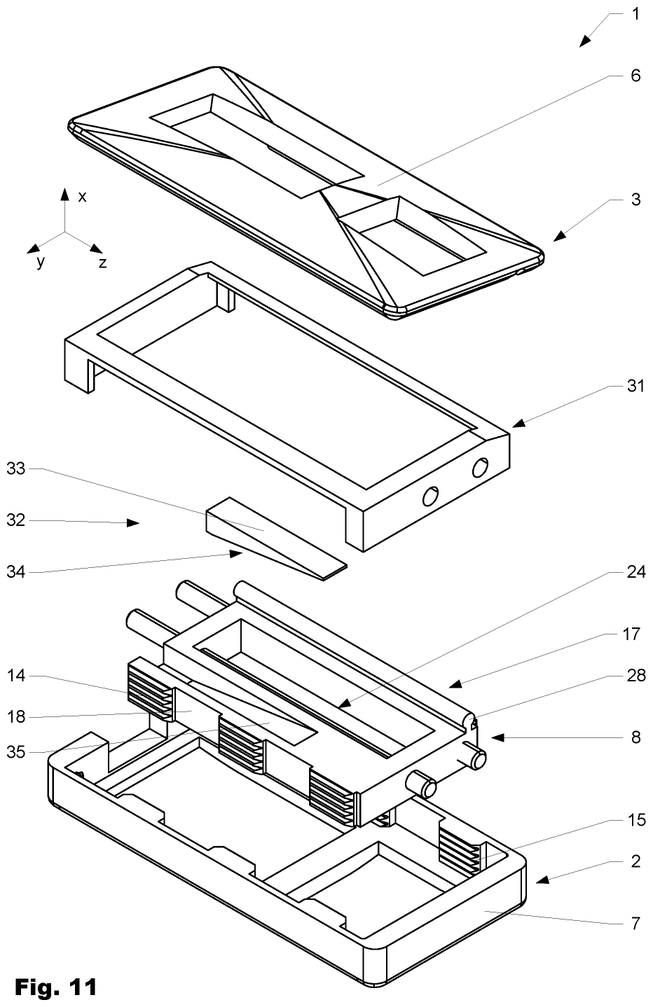

FIGS. 1-4 show a first variation an expandable intervertebral cage 1, which is not tiltable. FIGS. 5-9 show a second variation of an expandable intervertebral cage 1, FIG. 10 a third variation of an expandable intervertebral cage 1 and FIG. 11 a fourth variation of an expandable intervertebral cage 1. In the second, third and fourth variation the stage is tiltable with respect to the base.

In all variations, the expandable intervertebral cage comprises a base 2 and a thereto in a first direction (x) displaceable stage 3. The base 2 comprises a peripheral circumferential side wall 7 extending in the first direction and a first bone interaction surface 5, wherein the stage 3 comprising a second bone interaction surface 6 arranged essentially opposite to the first bone interaction surface 5 with respect to the expandable intervertebral cage 1. In order to lock the stage 3 with respect to the base 2 in the first direction respectively the position of the second bone interaction surface 6 of the stage 3 with respect to the first bone interaction surface 5 of the base 2, the cage comprises a locking mechanism 4 with at least one latch supporting the stage 3 in the locked position in the first direction. For a space saving design the locking mechanism 4 can be arranged fully within the circumferential side wall 7 of the base 2, as shown.

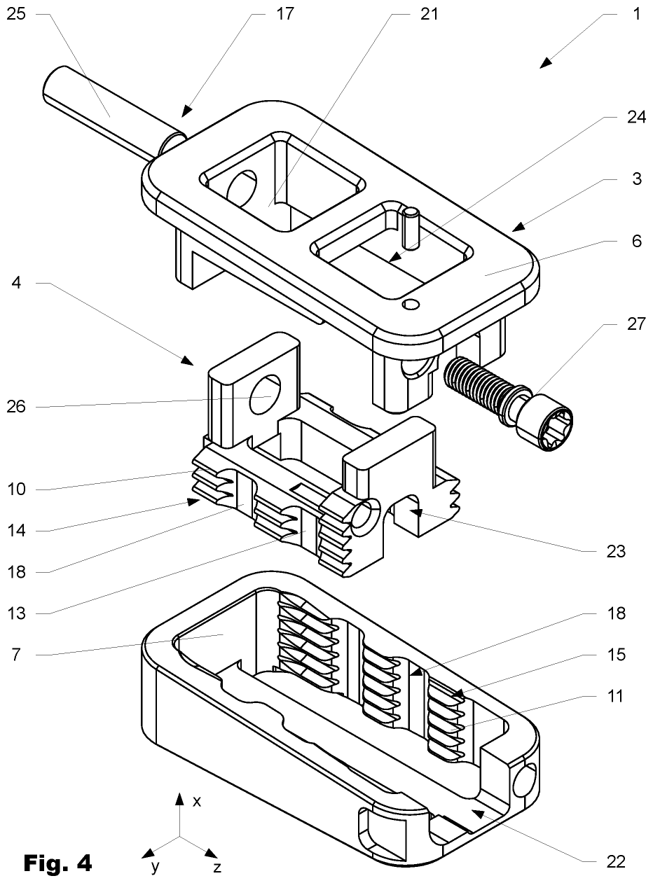

In the first variation shown in FIGS. 1-4, the locking mechanism 4 comprises one latch 8 arranged in a displaceable manner in a second direction (z) essentially perpendicular to the first direction (x) between a locked position and an unlocked position. In the locked position, the stage 3 is supported and locked in the first direction. In the unlocked position, the latch 8 is displaceable in the first direction with respect to the base 2. The latch 8 hereby comprises multiple first teeth 10 arranged in rows 14 on an outer side 13 of the latch 8. The multiple first teeth 10 of a row 14 are spaced apart from each other in the first direction, as can be seen in FIG. 4. Accordingly, the multiple second teeth 11 are arranged in rows 15 on an inner side 12 of the circumferential side wall 7 of the base 2. In the shown variation, six rows 14, 15 of respective first and second teeth 10, 11 are present. Thereby the rows of the second teeth 15 are arranged pairwise opposite of each other on the circumferential side wall 7, in particular on the part of the circumferential side wall 7 extending in the second direction (z). The rows of the first teeth 14 of the latch 8 are also arranged pairwise opposite of each other with respect to the latch 8. If the first and second teeth 10,11 are engaged, the latch 8 is latched is a locked position. By displacing the latch is the second direction, the first and second teeth disengage. In the unlocked position the rows of first teeth 14 are arranged in a respective groove 18 of the circumferential side wall 7 extending in the first direction. Thus, no undercut is formed between the latch 8 and the base 2 in the first direction.

As can be seen in FIG. 3, the latch 8 can be displaced by means of a screw 27 engaging with an internal thread 16 of the latch 8. The screw 27 extends through the stage 3 in the latch 8. Additionally, the latch 8 can be interconnected with the stage 3 by a linear guiding structure 17. In the shown variation the linear guiding structure 17 comprises a rod 25 mounted in the stage 3 and extending through an opening 26 of the latch 8 (compare FIG. 4). The rod 25 and the opening 26 extend in said second direction.

An expansion tool can be inserted in a tool opening 22 arranged between the base 2 and the stage 3. The tool supports and displaces the stage 3 with respect to the base 2 in the first direction in the unlocked position. If a desired expanded position is achieved, the latch 8 can again be moved in the locked position by rotating the screw 27. In the locked position the expansion tool can be removed.

In the first variation of the expandable intervertebral cage the first bone interaction surface 5 and the second bone interaction surface 6 are arranged at a fixed angle with respect to each other. Meanwhile the second, third and fourth variation of the expandable intervertebral cage as explained in more detail hereinafter, allows for various different angles between the first bone interaction surface 5 and the second bone interaction surface 6.

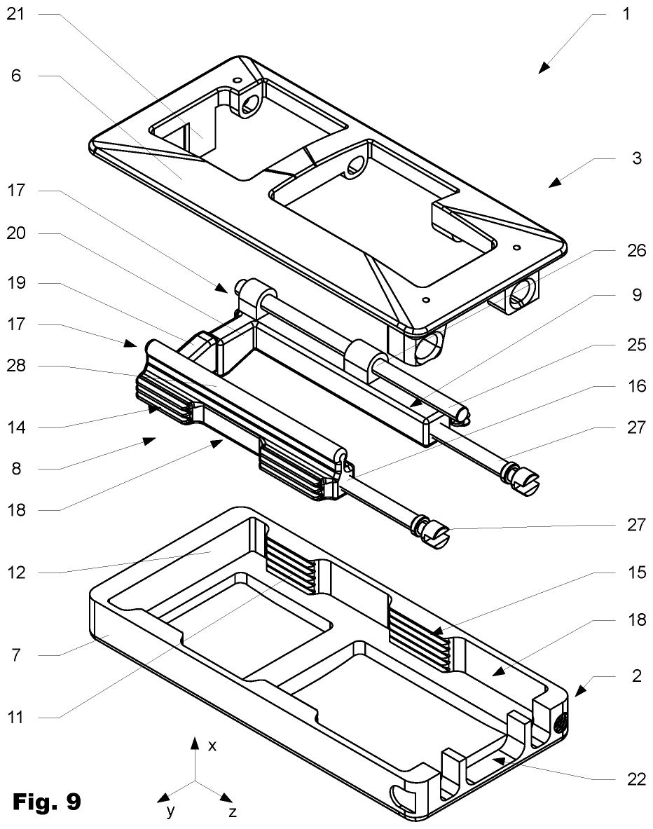

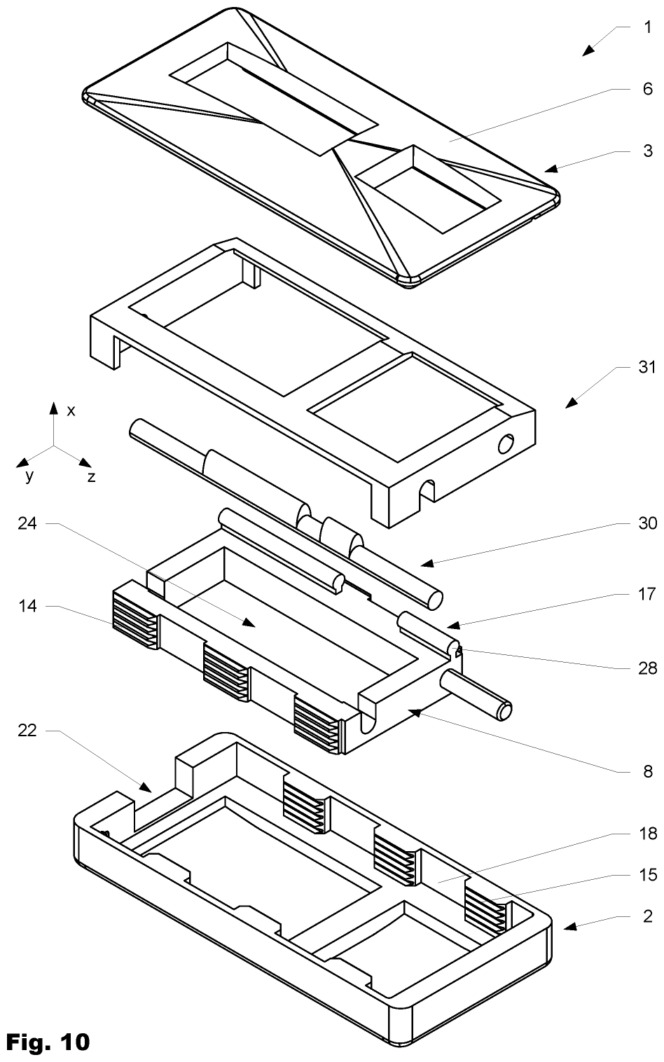

In the second variation as illustrated in FIG. 5-FIG. 9, the locking mechanism 4 therefore comprises a first and a second latch 8, 9 arranged in a displaceable manner in a second direction (z) essentially perpendicular to the first direction (x) between the locked position and the unlocked position. The second latch is hereby arranged independently displaceable from the first latch in the first and/or the second direction to allow to tilt the stage 3 with respect to the base 2 around the second direction (z). The second variation is suitable for LLIF applications. As can be seen in FIG. 9, the base 2 has (equivalently to the first variation) a peripheral circumferential side wall 7 extending in the first direction with a first bone interaction surface 5. On an inner side 12 of the circumferential side wall 7 four rows 15 of multiple second teeth 11 are arranged. The multiple second teeth 11 of each row 15 are spaced apart from each other in the first direction. A respective groove 18 is arranged adjacent to each row 15 extending also in the first direction. The rows 15 (and the grooves 18) are arranged pairwise opposite of each other in respect to the circumferential side wall 7. Also, the first and the second latch 8, 9 each comprises a number of rows 14 of first teeth 10, wherein the number of rows 14 of the first and the second latch 8, 9 in the shown variation is half of the number of rows 15 of second teeth 11. Each latch 8, 9 comprises a thread 16, which engages with a screw 27 extending in the second direction. By rotating the screws 27 with a tool, the respective latch 8, 9 is displaced in the second direction with respect to the base 2 thereby displacing the first teeth 10 from a locked position (where the rows of the first and second teeth 14, 15 are engaged) to an unlocked position (where the rows of first teeth 14, 15 are arranged in the grooves 18).

The cage further comprises per latch 8,9 a linear guiding structure 17, configured to support the displacement of the respective latch 8, 9 in the second direction, while positioning the respective latch 8, 9 in the first direction with respect to the stage 3. In case of the first latch 8, the linear guiding structure 17 comprises an (integral) omega-shaped guiding element 28 arranged on the first latch 8 and extending in the second direction. When mounted, the guiding element 28 is positioned and guided in a respective notch 29 of the stage 3 (compare e.g. FIG. 7). In case of the second latch 9, the linear guiding structure 17 comprises a rod 25, extending in the second direction through respective openings 26 of the second latch 9 and being mounted in the stage 3. Both variations of the linear guiding structure 17 allow a rotation around the second direction. The degree of freedom around the second direction is important for the second variation of the cage, since said cage can be tilted around the second direction in order to take the patients lumbar angle into account. During insertion of the cage between the vertebrae, the cage is in an initially retracted state. Once positioned between the vertebrae, the locking mechanism may be brought in an unlocked position and a tool can be inserted in a tool opening. The tool can be temporarily affixed during operation to the cage 1 by a tool interface 23. With the affixed tool, the stage 3 can be lifted in the first direction and tilted around the second direction with respect to the base 2. Once a desired expanded state of the stage 3 is found, the screws 27 can be rotated such that the first and second latch 8, 9 are displaced in the second direction and the teeth once again engage in the locked position. As can be been in FIG. 7, the first latch 8 further comprises a first support 19 interacting in the locked position with a second support 20 of the second latch 9. The first and the second support are braced against each other in the third direction (y).

In the first and second variation, the cage 1 further comprises a strut 21 extending in a mounted position from the stage 3 into the circumferential side wall 7 of the base 2 in order to prevent unwanted displacement in the second direction and/or unwanted tilting around the third direction. Both variation further feature a filling room 24 for accommodating bone graft. The filling room 24 can be accessed through an opening in the first bone interaction surface 5 and/or the second bone interaction surface 6.

In the third variation as illustrated in FIG. 10, the locking mechanism 4 comprises only one latch 8, as explained in context with the first variation of the cage 1. However, in difference to the first variation, the cage 1 further comprises a camshaft 30 and a mounting frame 31 to facilitate tilting of the stage 3 with respect to the base 2. As can be seen, the camshaft 30 extends in the second direction (z) and is mounted between the latch 8 and the mounting frame 31. In the shown variation, the linear guiding structure 17 comprises an (integral) omega-shaped guiding element 28 arranged on the latch 8 and extending in the second direction. When mounted, the guiding element 28 is positioned and guided in a respective notch of the stage 3. Hereby, the linear guiding structure 17 allows a rotation around the second direction, such that if the camshaft 30 is rotated and the stage 3 is locally lifted by the cam of the camshaft 30, the stage 3 can be tilted around the second direction with respect to base 2 in order to take the patients lumbar angle into account.

In the fourth variation as illustrated in FIG. 11, the cage 1 features a latch 8 and a looking mechanism, as explained in context with the first variation of the cage 1. However, in difference to the first variation, the cage 1 further comprises a wedge 32 to facilitate tilting of the stage 3 with respect to the base 2. The wedge 32 is arranged in the first direction (x) between the stage 3 and the latch 8. A first gliding surface 33 of the wedge 32 interacts thereby with a contact surface of the stage 3 and second gliding surface 34 of the wedge 32 interacts with a contact surface 35 of the latch 8. Thus, by displacing the wedge 32 in the second direction (z) with a tool, the stage 3 is lifted one sided from the base 2 in the first direction resulting in a tilting movement of the stage 3 with respect to the base 2. In the shown variation, the linear guiding structure 17 also comprises an omega-shaped guiding element 28 arranged on the first latch 8 and extending in the second direction and allowing a rotation around the second direction, such that if the wedge 32 is displaced and the stage 3 locally lifted, the stage 3 can be tilted around the second direction with respect to base 2. The words used in the specification are words of description rather than limitation, and it is understood that various changes may be made without departing from the scope of the invention.

1. An expandable intervertebral cage (1) comprising:
a. a base (2) comprising a peripheral circumferential side wall (7) extending in a first direction (x) and a first bone interaction surface (5);
b. a stage (3) comprising a second bone interaction surface (6) arranged essentially opposite to the first bone interaction surface (5) with respect to the expandable intervertebral cage (1); and
c. a locking mechanism (4) adapted to lock the position of the second bone interaction surface (6) of the stage (3) with respect to the first bone interaction surface (5) of the base (2) at least in the first direction (x) and arranged at least partially within the circumferential side wall (7) of the base (2).

2. The expandable intervertebral cage (1) according to claim 1, wherein the locking mechanism (4) comprises at least one latch (8, 9) arranged in a displaceable manner in a second direction (z) essentially perpendicular to the first direction (x) between:
a. a in the first direction locked position, in which the stage (3) is supported in the first direction (x), and
b. a in the first direction (x) unlocked position, in which the at least one latch (8) is displaceable in the first direction (x) with respect to the base (2).

3. The expandable intervertebral cage (1) according to claim 2, wherein the at least one latch (8, 9) comprises at least one first tooth (10), which is configured to latch in the locked position with at least one second tooth (11) arranged at the base (2).

4. The expandable intervertebral cage (1) according to claim 3, wherein at least one row of multiple first teeth (14) is arranged on an outer side (13) of the at least one latch (8,9), wherein the multiple first teeth (10) of the respective row (14) are spaced apart from each other in the first direction (x).

5. The expandable intervertebral cage (1) according to claim 3, wherein at least one row of multiple second teeth (15) are arranged on an inner side (12) of the circumferential side wall (7), wherein the multiple second teeth (11) of each row (15) are spaced apart from each other in the first direction (x).

6. The expandable intervertebral cage (1) according to claim 2, wherein the at least one latch (8, 9) is arranged displaceable in the second direction (z) by a thread (16).

7. The expandable intervertebral cage (1) according to claim 2, wherein the expandable intervertebral cage (1) comprises a linear guiding structure (17) interconnecting the at least one latch (8,9) to the stage (3) and configured to support the displacement of the at least one latch (8,9) in the second direction (z).

8. The expandable intervertebral cage (1) according to claim 2, wherein the expandable intervertebral cage (1) comprises a first latch (8) and second latch (9) arranged independently displaceable with respect to each other in the first and/or the second direction (x, z).

9. The expandable intervertebral cage (1) according to claim 8, wherein the first latch (8) and the second latch (9) support each other in a third direction (y) in the locked position.

10. The expandable intervertebral cage (1) according to claim 1, wherein the expandable intervertebral cage (1) comprises a strut (21) extending in a mounted position from the stage (3) into the circumferential side wall (7), such that unwanted displacement of the base (2) with respect to the stage (3) in the second direction (z) is prevented.

11. The expandable intervertebral cage (1) according to claim 1, wherein the expandable intervertebral cage (1) comprises a rotatable camshaft (30) or a displaceable wedge (32) for locally lifting the stage (3) with respect to the base (2) such that the stage (3) is tilted with the respect to the base (2).

12. The expandable intervertebral cage (1) according to claim 11, wherein the cam shaft (30) or the wedge (32) is arranged between the stage (3) and the at least one latch (8) in the first direction.

13. The expandable intervertebral cage (1) according to claim 1, wherein a tool opening (22) is arranged between the base (2) and the stage (3) configured to receive a tool to displace the stage (3) with respect to the base (2) in the first direction (x).

14. The expandable intervertebral cage (1) according to claim 13, wherein the base (2) and/or the stage (3) comprise a tool interface (23) for temporarily affixing the tool during operation.

15. The expandable intervertebral cage (1) according to claim 1, wherein the first bone interaction surface (5) and the second bone interaction surface (6) are arranged at an angle with respect to each other.

16. The expandable intervertebral cage (1) according to claim 1, wherein the at least one latch is framelike circumventing a filling room (24) for accommodating bone graft.
